(19) United States
(12) Patent Application Publication
(10) Pub. No.: US 2005/0228333 A1
(43) Pub. Date: Oct. 13, 2005

(54) Method of stimulating blood-derived components using no added thrombin or other agonist

(76) Inventors: Calvin Britton, Little Rock, AR (US); Carl Keller, Little Rock, AR (US)

Correspondence Address: MARK MURPHEY HENRY, 204 SOUTH EAST AVENUE, FAYETTEVILLE, AR, 72701, US

(21) Appl. No.: 10/821,747

(22) Filed: Apr. 8, 2004

(51) Int. Cl. A61M 37/00

(52) U.S. Cl. 604/4.01

(57) ABSTRACT

The present disclosure contemplates the creation of an active turgid gas liquid interface as means to stimulate various blood components contained within a blood sample, thereby facilitating the formation of fibrin contained within the blood sample, thereby increasing the viscosity of the sample, with such sample being applied to damaged tissue and facilitating tissue repair or tissue sealing components.

### CROSS REFERENCES

None.

### GOVERNMENT RIGHTS

None.

### BACKGROUND OF THE DISCLOSURE

In the field wound care, it is a known strategy to separate whole blood into various sub-components and to apply stimulated sub-components to damaged tissue in an effort to accelerate, augment, or effectuate the tissue repair, closure, and healing process. It is generally understood that the whole blood is separated by centrifugation, sequestration, filtration, or other mechanical process such that at least three dominant components are isolated based on molecular weight or size. At least three components are understood to result from such traditional separation, including but not limited to red blood cells, platelet-poor plasma, and platelet-rich plasma. The platelet-rich plasma may comprise platelets, white blood cells, fibrinogen, plasma, stem cells and plasma proteins.

Generally, in connection with creating a tissue sealant and/or filler for use in acute or chronic wound healing and damaged tissue repair, the prior art attempts to activate the platelet rich plasma or the platelet-poor plasma portions by saturating either the platelet rich or platelet poor plasma portion with significant amounts of bovine-derived thrombin, collagen, serotonin, or other agonist. Following such activation, it is known that a variety of cellular pathways are triggered in such a way as to inevitably increase the viscosity of the plasma portion(s). For example, it is understood that, as part of these pathways and following the introduction of an agonist, fibrinogen present in the plasma portion will transform into fibrin. There is substantial evidence in the prior art that bovine-derived thrombin is applied in copious amounts in order to activate the respective plasma portions and/or platelet concentrates. For example, U.S. Pat. No. 6,524,568 indicates a range between 100 U and 10,000 U exogenous thrombin as mixed with an 8 mL platelet concentrate volume, and the patent claims a preferred amount of 1000 U thrombin per 8 ml of platelet concentrate.

The use of bovine or non-human-derived products is a widely used phenomenon. All this may be in accepted practice today, yet the inventor understands that it is best to significantly depart from the use of bovine-derived products in connection with the unsafe circumstances surrounding bovine spongiform encephalitis, known as BSE. As such, because it is very difficult at this point in time to even test for infection of BSE, the inventor invested substantial time and effort into formulating a method and procedure whereby no exogenously-applied thrombin or similar agonist is utilized. Obviously, because thrombin is endogenous to mammals, the inventor makes clear that this patent does not seek to neutralize or vitiate endogenous thrombin.

Thus, while exogenous thrombin is the current protocol in activating either platelet-rich plasma or platelet-poor plasma, the disclosure herein serves to satisfy in part, the goal of eliminating the reliance on super-saturation levels of exogenously applied thrombin, be the source of such thrombin derived from bovine sources or the product of concentration of the patient's own thrombin. It is believed that the reduction in exogenously-applied thrombin will translate into elevated confidence in certain medical procedures, easier compliance with federal regulations governing exogenously applied chemicals in the health care industry, and decreased cost in obviating the need for high-cost purification of bovine-derived thrombin or similar agonist.

### SUMMARY

This disclosure relates to the stimulation of blood plasma portions by creating a steady rate turgid gas and plasma portion interface. By creating a turgid gas/liquid interface, under controlled conditions, this disclosure thus seeks to activate the biological components associated with tissue repair and wound care in a manner that reduces exogenous chemical contact or treatments. In application, the inventor contemplates stimulating platelet rich plasma by careful percolation or injection of oxygen gas through an amount of platelet-rich plasma, although the same principles apply in either platelet-poor plasma or with whole blood as well.

In the first preferred embodiment discussed below, no exogenous thrombin was required to stimulate a sample of platelet rich plasma and corresponding platelets and other factors present within the platelet rich plasma.

It is therefore an object of the present disclosure to provide a combination comprising a therapeutic amount of autologous platelet-rich plasma that utilizes no exogenous thrombin yet nonetheless facilitates tissue sealing, repair, healing, and wound closure.

It is still further object of the present disclosure to provide an efficient method to stimulate platelet rich plasma by using steady gas percolation as means to create an active turgid gas/liquid interface, which obviously differs from the resting state gas/liquid interface occuring when the platelet rich plasma is simply exposed to ambient air in an open container.

It is still further object of the present disclosure to provide a preparation of concentrated platelet-rich plasma using an apparatus that permits ease of application of stimulated platelet-rich plasma to damaged tissue.

It is still further object of the present disclosure to provide a preparation of autologous platelet-rich plasma in a clinical environment to permit patients who experience acute, chronic, or recurrent wound procedures. Such benefiting procedures would include, but not be limited to, diabetic ulcers, venous, decubitus, surgical dehiscences wounds, bone repair and tissue remodeling in autologous wound care.

Towards the fulfillment of these and other objects and advantages, the present method relates to a first step of isolating from the patient an amount of whole blood and subjecting the whole blood to treatment with an anti-coagulant agent, followed by a centrifugation or separation process to obtain an amount of platelet-rich plasma. The second step comprises adding an effective amount of anti-coagulant neutralizing reagent. The third step comprises stimulating the platelet-rich plasma by creating a turgid gas/liquid interface. The platelet rich plasma, once properly stimulated following the creation of the turgid gas/liquid interface, will adopt certain characteristics such as increased viscosity and fibrin formation. The fourth step comprises applying the stimulated platelet rich plasma to, or infused within, damaged tissue.

### DESCRIPTION

The first preferred embodiment discussed in more detail below represents a process wherein the first step comprises isolating from the patient whole blood using venipuncture. As part of this isolation, it is preferable to receive the whole blood in a container that is treated with an effective amount of anti-clotting agent such as sodium citrate. Using platelet pheresis equipment, blood sequestration or separation mechanisms, the whole blood is thereafter centrifuged or otherwise processed and thereby separated into generally distinct components; i.e., the platelet-rich plasma, the platelet-poor plasma, and the red blood cell concentrates.

To initiate the second step, the technician or apparatus isolates the platelet-rich plasma and combines an effective amount of neutralizing agent to counter the effects of the anticlotting agent. Calcium chloride is an effective anti-coagulant neutralizing agent, although other agents may be used interchangeably.

The third step involves creation of a steady turgid gas/liquid interface by way of percolating through the platelet-rich plasma a steady stream of gas which in turn stimulates the platelet-rich plasma and triggers the transformation of fibrinogen to fibrin. Within a relatively brief period of time, the viscosity of the platelet-rich plasma will increase. Variability in stimulation created by the turgid gas/liquid interface depends upon the volume of platelet-rich plasma when compared to the size of the gas bubbles and the relative speed and rate of percolation of gas through the platelet-rich plasma, although there are generalized parameters, exampled generally thorough the Examples recited herein.

Following the stimulation of the platelet rich plasma, the platelet rich plasma will become viscous and gelatin-like. Once the platelet rich plasma becomes viscous, it is generally known as a tissue graft. As the fourth step, the tissue graft may then be molded, sculpted, or crafted to fit within, applied to, co-saturated with dried or donor material(s), induced into or applied around various grafts, appliances, tools, apparatus, or other fixtures or dressings used with bone or soft tissue repair, remodeling, sealing or healing a particular wound or tissue injury site or to fill surgical incisions.

It is generally desirable for platelet rich plasma, once initially stimulated, to transform into a tissue graft in not more than fifteen minutes. Using the process described herein, the desired viscosity of the tissue graft was reliably, and consistently, obtained in less than fifteen minutes. This time period is acceptable for the industry. In fact, using exogenously-applied thrombin concentrations otherwise referenced in U.S. Pat. No. 6,524,568, the formation of the viscous platelet graft occurred also within 15 minutes. Of course, the variability associated with the time it takes for the platelet rich plasma to become first stimulated and when the platelet-rich plasma changes viscosity and form a tissue graft varies from patient to patient, and one cause for such variability appears to be a function of the fibrinogen or platelet levels of the patient. When using the steady percolation method, another source of variability appears to be a function of the size of the gas bubbles and the rate, platelet rich plasma (“PRP”) volume, and speed of percolation.

In a second preferred embodiment, the goal is to simply remove from the whole blood the majority of red blood cells. It is the inventor's experience that the introduction of red blood cells into a wound exacerbates wound healing. For that reason, the second preferred embodiment contemplates the use of that portion of plasma, platelets, fibrinogen, white blood cells, and other cellular structures, as long as the number of red blood cells is reduced when compared to whole blood. Existing technology permits easy isolation of red blood cells, so this disclosure does not contemplate any one mode of centrifugation, sequestration, filtration or separation process over another; instead, this disclosure contemplates a need to separate out red blood cells from the whole blood to decrease the ill-effects associated with degredation of red blood cells within such damaged tissue once the stimulated tissue graft is applied to the damaged tissue. The activation process in the second preferred embodiment is disclosed in the first preferred embodiment.

### EXAMPLE 1

Whole blood was collected from the antecubital vein in the arm into a container with an appropriate amount of anticoagulant agent, sodium citrate, and processed by centrifugation to sequester platelet rich plasma. The platelet rich plasma was combined with 0.05 cc 10% CaCl per 1 cc of platelet rich plasma in order to neutralize the effects of the anticoagulant. The platelet rich plasma was then gently and steadily bubbled (10 bubbles per second) with Oxygen gas to stimulate the platelet rich plasma. The gas was percolated for fifteen minutes or until the platelet rich plasma converted from a liquid form into a substantially gelatinous form. This entire transformation generally takes less than fifteen (15) minutes. The size of the bubble was an estimated 4 mm in diameter.

### EXAMPLE 2

Using the same procedure in Example 1 to isolate platelet rich plasma, and thereafter treating the platelet rich plama with anti-coagulant neutralizer, the platelet rich plasma was gently and steadily bubbled (1 bubble per second) with Oxygen gas to stimulate the platelet rich plasma. The gas was percolated for up to fifteen (15) minutes, until the platelet rich plasma converted from a liquid form into a substantially gelatinous form. The size of the bubble was an estimated 4 mm in diameter.

### EXAMPLE 3

Using the same procedure in Example 1 to isolate platelet rich plasma and thereafter neutralize the anti-coagulant, the platelet rich plasma was steadily bubbled using a rolling bubble stream (15-50 bubbles per second) with Oxygen gas to stimulate the platelet rich plasma. The gas was initially percolated for two (2) minutes using this rolling bubble stream and then removed, permitting the platelet rich plasma to sit idle in order to facilitate opportunity for the blood components to build the necessary latticework and structural cross-linking and become more viscous. This entire transformation generally takes approximately ten (10) minutes. The size of the bubble was an estimated 4 mm in diameter, although bubbles as large as 1 cm have proven successful.

Under the three above examples, the tissue graft is uniform across all surfaces and throughout.

### EXAMPLE 4

Whole blood was collected from the antecubital vein in the arm into a container with an appropriate amount of anticoagulant agent, sodium citrate, and processed by centrifugation to sequester primarily platelet rich plasma. The platelet rich plasma was combined with 0.05 cc 10% CaCl per 1 cc of platelet rich plasma in order to neutralize the effects of the anticoagulant. The platelet rich plasma was then gently and steadily bubbled (10 bubbles per second) with Nitrogen gas to stimulate the platelet rich plasma. The gas was percolated for three minutes or until the platelet rich plasma converted from a liquid form into a substantially gelatinous form. This entire transformation generally takes less than fifteen (15) minutes. The size of the bubble was an estimated 4 mm in diameter.

### EXAMPLE 5

Using the same procedure in Example 1 to isolate platelet rich plasma and thereafter neutralize the anti-coagulant, the platelet rich plasma was thereafter divided into two equal quantities and placed in two equal glass beakers, such beakers being designated “first beaker” and the second designated “second beaker.” The first beaker was percolated with Oxygen gas at a rate of approximately 5 bubbles per second for 13 minutes, and the platelet rich plasma in the first beaker thereafter formed a viscous and expected graft material. Over the same duration, the second beaker, exposed simply to ambient air, showed no signs of stimulation, and there were no visible clots or increased viscosity.

The foregoing examples do not necessarily limit the scope of the disclosure herein, and it is only provided to establish actual step-by-step methods by which the invention herein can be utilized effectively to achieve platelet rich plasma gels without exogneous application of thrombin or other agonist.

1. A method comprising:
(a) isolating from mammalian blood one or more of its constituents to form an isolate,
(b) percolating gas bubbles through said isolate thereby stimulating at least one blood constituent situated therein and, thereafter,
(c) applying said isolate to damaged tissue to facilitate tissue repair.

2. The method of claim 1 wherein:
at step (b), said gas bubbles comprises Oxygen.

3. The method of claim 1 wherein:
at step (b), said gas bubbles comprises Nitrogen.

4. The method of claim 1 wherein:
at step (b), said bubbles comprise an inert gas.

5. The method of claim 1 wherein:
at step (b), said gas bubbles comprise a non-toxic gas.

6. A method for activating platelet rich plasma comprising:
(a) isolating from whole blood an amount of platelet rich plasma;
(b) percolating gas bubbles through said platelet rich plasma to stimulate at least one blood component situated therein;
(c) applying the stimulated platelet rich plasma to damaged tissue to facilitate tissue repair.

7. The method of claim 6 wherein:
at step (b), said gas bubbles comprises Oxygen.

8. The method of claim 6 wherein:
at step (b), said gas bubbles comprises Nitrogen.

9. The method of claim 6 wherein:
at step (b), said bubbles comprise an inert gas.

10. The method of claim 6 wherein:
at step (b), said gas bubbles comprise a non-toxic gas.

11. A method for activating platelet poor plasma comprising:
(a) isolating from whole blood an amount of platelet poor plasma;
(b) percolating gas bubbles through said platelet poor plasma to stimulate at least one blood component situated therein;
(c) applying the platelet poor plasma to damaged tissue to facilitate tissue sealing, repair, or both.

12. The method of claim 11 wherein:
at step (b), said gas bubbles comprises Oxygen.

13. The method of claim 11 wherein:
at step (b), said gas bubbles comprises Nitrogen.

14. The method of claim 11 wherein:
at step (b), said bubbles comprise an inert gas.

15. The method of claim 11 wherein:
at step (b), said gas bubbles comprise a non-toxic gas.
